(19) United States
(12) Patent Application Publication
(10) Pub. No.: US 2026/0076709 A1
(43) Pub. Date: Mar. 19, 2026

(54) BALLOON FOR BALLOON CATHETER AND BALLOON CATHETER INCLUDING SAME

(71) Applicant: KANEKA CORPORATION, Osaka (JP)

(72) Inventors: Masahiro Kojima, Osaka (JP); Takahisa Hamabuchi, Osaka (JP); Masato Tsueda, Osaka (JP)

(73) Assignee: KANEKA CORPORATION, Osaka (JP)

(21) Appl. No.: 19/398,274

(22) Filed: Nov. 24, 2025

Related U.S. Application Data
(63) Continuation of application No. PCT/JP2024/018121, filed on May 16, 2024.

(30) Foreign Application Priority Data
May 30, 2023 (JP) 2023-089136

(51) Int. Cl. A61B 17/3207; A61M 25/10

(52) U.S. Cl. CPC A61B 17/320725; A61M 2025/1004; A61M 25/104; A61M 2025/109

(57) ABSTRACT

A balloon for a balloon catheter that, when deflated, can cut a stenosed site and be easily folded to reduce outer diameter and a balloon catheter including the balloon are provided.

The balloon includes a balloon main body having an outer surface and an inner surface and a protruding portion protruding outward in a radial direction from the outer surface of the main body and extending in a longitudinal axis direction. The protruding portion in a straight tube portion has an ST-portion notch and the protruding portion in a distal-side tapered portion has a DT-portion notch. A depth of the ST-portion notch and a depth (D4) of the DT-portion notch are each 50 μm or more, and the depth (D3), a height (H3) of the protruding portion in the straight tube portion, the depth (D4), and a height (H4) of the protruding portion in the distal-side tapered portion satisfy D3/H3≤D4/H4.

### TECHNICAL FIELD

One or more embodiments of the present invention relate to a balloon for a balloon catheter and a balloon catheter including the same.

### BACKGROUND

Angioplasty, in which a balloon catheter is inserted into a stenosed site of a blood vessel and the balloon is inflated to expand the blood vessel and secure blood flow, is widely performed as a minimally invasive therapy. Angioplasty is used, for example, for the treatment of diseases such as myocardial infarction caused by stenosis occurring in the coronary artery of the heart, the treatment of stenosis occurring in a shunt portion for dialysis, and the like.

In angioplasty, it is sometimes difficult to expand a stenosed site hardened by calcification or the like with a general balloon catheter. In addition, a method of expanding a stenosed site by placing an indwelling expansion instrument called a stent in the stenosed site is also used. However, for example, a lesion such as an in-stent restenosis (ISR) lesion in which neointima of a blood vessel excessively proliferates after the treatment and stenosis of the blood vessel occurs again may occur. In the ISR lesion, since the neointima is soft and the surface thereof is slippery, in a general balloon catheter, the position of the balloon may be shifted from the lesion portion at the time of expanding the balloon, and the blood vessel may be damaged.

As a balloon catheter capable of expanding a stenosed site even in such a calcified lesion or an ISR lesion, a balloon catheter in which a protruding portion, a blade, or a scoring element for biting into a stenosed site is provided on a balloon has been developed. For example, PTL 1 discloses a balloon catheter including a scoring element made of a polymer material having higher rigidity than a polymer material forming a balloon main body, in which the scoring element is flattened at one end and the other end of the balloon. PTL 2 discloses a scoring balloon structure in which the height of a scoring element decreases in accordance with the tapered shape of a balloon, and PTL 3 discloses a balloon catheter in which an outer protruding portion is provided in a straight tube portion of a balloon and in which an inner protruding portion is provided in a tapered portion. In the above-mentioned PTLs 1 to 3, the height of the scoring element is reduced at both ends of the balloon, or the inner protruding portion is provided instead of the outer protruding portion. In contrast, there is also a balloon catheter in which the amount of protruding portion of a protruding portion disposed in a distal-side tapered portion of a balloon is larger than that of a protruding portion disposed in a straight tube portion (PTL 4).

### PATENT LITERATURE

PTL 1: U.S. Patent Application Publication No. 2016/0128718
PTL 2: Japanese Translation of PCT International Application Publication No. 2014-506140
PTL 3: International Publication No. 2020/012851
PTL 4: International Publication No. 2020/012850

A balloon catheter is inserted into a body cavity and delivered to a treatment site while it is in a deflated and folded state. Therefore, in each of the balloon catheters disclosed in the above-mentioned PTLs 1 to 3, an attempt has been made to improve the maneuverability of the balloon by reducing the height of the scoring element at a distal end portion of the balloon so as to facilitate insertion into a body cavity, thereby suppressing an increase in the outer diameter. Further, in the balloon catheter disclosed in PTL 4, the height of the protruding portion disposed in the distal-side cone region is made high such that, when only the distal-side cone region is introduced into a lesion portion and the balloon is inflated, the balloon can be inflated while making a cut into the lesion portion with an element provided in the distal-side cone region. However, none of these balloons has been intended to be used to cut a stenosed site while the balloon is in a deflated state and is being advanced or retracted.

### SUMMARY

In view of the above-described circumstances, a balloon for a balloon catheter that includes a protruding portion formed on a surface thereof, that is capable of cutting a stenosed site while the balloon is in a deflated state, that is capable of improving a function of expanding a stenosed site while the balloon is in an inflated state, and that can be easily folded when deflated, thereby narrowing the outer diameter thereof and improving the maneuverability thereof, and a balloon catheter including the balloon are provided.

A balloon for a balloon catheter according to one or more embodiments of the present invention that can address the above is as follows.
[1] A balloon for a balloon catheter comprising: a straight tube portion; a proximal-side tapered portion positioned on a proximal side of the straight tube portion; a proximal-side sleeve portion positioned on a proximal side of the proximal-side tapered portion; a distal-side tapered portion positioned on a distal side of the straight tube portion; a distal-side sleeve portion positioned on a distal side of the distal-side tapered portion; a balloon main body having an outer surface and an inner surface; and a protruding portion protruding radially outward from the outer surface of the balloon main body and extending in a longitudinal axis direction. The protruding portion in the straight tube portion has at least one ST-portion notch, and the protruding portion in the distal-side tapered portion has at least one DT-portion notch. A depth D3 of the at least one ST-portion notch and a depth D4 of the at least one DT-portion notch are both 50 μm or more. The depth D3 of the at least one ST-portion notch, a height H3 of the protruding portion in the straight tube portion, the depth D4 of the at least one DT-portion notch, and a height H4 of the protruding portion in the distal-side tapered portion satisfy a relationship of D3/H3≤D4/H4.

The protruding portion is disposed in the distal-side tapered portion at which the outer diameter of the balloon main body in an inflated state gradually decreases from the straight tube portion, and thus, the protruding portion in the distal-side tapered portion is easily exposed beyond a wing when the balloon is deflated, thereby being capable of contributing to cutting of a stenosed site. In this case, by advancing the balloon while keeping it deflated (inching the balloon forward), the stenosed site can be cut. The value of the ratio D4/H4 of the depth D4 of the at least one DT-portion notch of the protruding portion in the distal-side tapered portion to the height H4 of the protruding portion in the distal-side tapered portion is relatively large, and thus, each segment of the protruding portion divided by the at least one DT-portion notch in the distal-side tapered portion can act on the stenosed site, thereby improving the effect of making a cut in the stenosed site by an edge formed by a boundary portion between the protruding portion and the at least one DT-portion notch and the effect of scraping the stenosed site. Therefore, by performing an operation such as inching the balloon forward while the balloon is deflated, the stenosed site can be expanded by the protruding portion in the distal-side tapered portion. Further, the value of the ratio D3/H3 of the depth D3 of the at least one ST-portion notch of the protruding portion in the straight tube portion to the height H3 of the protruding portion in the straight tube portion is relatively small, and thus, the rigidity of the protruding portion in the straight tube portion can be improved, so that, when the balloon is inflated such that the protruding portion in the straight tube portion is exposed beyond the wing, the function of expanding the stenosed site by the protruding portion in the straight tube portion can be improved. Further, since the value of the ratio D3/H3 is relatively small, when the balloon is deflated and the wing is folded, it is possible to prevent the wing from being caught by a notch in the straight tube portion. As a result, the wing can be easily folded to reduce the outer diameter of the balloon, and thus, the maneuverability of the balloon can be improved when the balloon is deflated and removed from the body or when the balloon is reinserted into another treatment site.

A balloon for a balloon catheter according to one or more embodiments of the present invention may be any one of the following [2] to [16].
[2] The balloon for a balloon catheter according to [1], wherein the at least one ST-portion notch includes a plurality of ST-portion notches, and the depth D3 is an average of depths of the plurality of ST-portion notches.
[3] The balloon for a balloon catheter according to [1] or [2], wherein the at least one DT-portion notch includes a plurality of DT-portion notches, and the depth D4 is an average of depths of the plurality of DT-portion notches.
[4] The balloon for a balloon catheter according to any one of [1] to [3], wherein a height H3 of the protruding portion in the straight tube portion and a height H4 of the protruding portion in the distal-side tapered portion satisfy a relationship of H3≥H4.
[5] The balloon for a balloon catheter according to any one of [1] to [4], wherein the protruding portion in the proximal-side tapered portion has at least one PT-portion notch, a depth D2 of the at least one PT-portion notch is 50 μm or more, and the depth D3 of the at least one ST-portion notch, a height H3 of the protruding portion in the straight tube portion, the depth D2 of the at least one PT-portion notch, and a height H2 of the protruding portion in the proximal-side tapered portion satisfy a relationship of D3/H3≤D2/H2.
[6] The balloon for a balloon catheter according to [5], wherein the at least one PT-portion notch includes a plurality of PT-portion notches, and the depth D2 is an average of depths of the plurality of PT-portion notches.
[7] The balloon for a balloon catheter according to [5] or [6], wherein a height H3 of the protruding portion in the straight tube portion and the height H2 of the protruding portion in the proximal-side tapered portion satisfy a relationship of H3≥H2.
[8] The balloon for a balloon catheter according to [5] or [6], wherein a height H3 of the protruding portion in the straight tube portion and the height H2 of the protruding portion in the proximal-side tapered portion satisfy a relationship of H3<H2.
[9] The balloon for a balloon catheter according to any one of [1] to [4], wherein the protruding portion in the proximal-side tapered portion has a PT-portion notch, a depth D2 of the PT-portion notch is 50 μm or more, and the depth D3 of the at least one ST-portion notch, a height H3 of the protruding portion in the straight tube portion, the depth D2 of the PT-portion notch, and a height H2 of the protruding portion in the proximal-side tapered portion satisfy a relationship of D3/H3>D2/H2.
[10] The balloon for a balloon catheter according to [9], wherein a height H3 of the protruding portion in the straight tube portion and the height H2 of the protruding portion in the proximal-side tapered portion satisfy a relationship of H3≥H2.
[11] The balloon for a balloon catheter according to [9], wherein a height H3 of the protruding portion in the straight tube portion and the height H2 of the protruding portion in the proximal-side tapered portion satisfy a relationship of H3<H2.
[12] The balloon for a balloon catheter according to any one of [1] to [11], wherein a depth D3 of the at least one ST-portion notch and a height H3 of the protruding portion in the straight tube portion satisfy a relationship of D3≥H3×¼.
[13] The balloon for a balloon catheter according to any one of [1] to [12], wherein the depth D4 of the at least one DT-portion notch and a height H4 of the protruding portion in the distal-side tapered portion satisfy a relationship of D4≥H4×¼.
[14] The balloon for a balloon catheter according to any one of [1] to [13], wherein the protruding portion in the proximal-side tapered portion has a PT-portion notch, a depth D2 of the PT-portion notch is 50 μm or more, and the depth D2 of the PT-portion notch and a height H2 of the protruding portion in the proximal-side tapered portion satisfy a relationship of D2≥H2×¼.
[15] The balloon for a balloon catheter according to any one of [1] to [14], wherein the at least one ST-portion notch is greater in number than the at least one DT-portion notch.
[16] The balloon for a balloon catheter according to any one of [1] to [15], wherein the protruding portion in the proximal-side tapered portion has a PT-portion notch, a depth D2 of the PT-portion notch is 50 μm or more, and the at least one ST-portion notch is greater in number than the PT-portion notch.

One or more embodiments of the present invention also provide the following.
[17] A balloon catheter including the balloon for a balloon catheter according to any one of the above [1] to [16].

According to the above-described balloon for a balloon catheter and the balloon catheter including the same, the value of the ratio D4/H4 of the depth of the notch of the protruding portion in the distal-side tapered portion to the height of the notch is relatively large, and thus, the effect of making a cut in a stenosed site by the edge formed by the boundary portion between the protruding portion and the notch and the effect of scraping the stenosed site can be improved. Therefore, by performing an operation such as inching the balloon forward while the balloon is deflated, the stenosed site can be expanded by the protruding portion in the distal-side tapered portion while the balloon is deflated. In addition, the value of the ratio D3/H3 of the depth of the notch of the protruding portion in the straight tube portion to the height of the notch is relatively small, and thus, the rigidity of the protruding portion in the straight tube portion can be improved, so that the function of expanding the stenosed site while the balloon is inflated can be improved. Further, since the value of the ratio D3/H3 is relatively small, the wing can be easily folded when the balloon is deflated, and this facilitates removal of the balloon from the body and reinsertion of the balloon into another treatment site.

### BRIEF DESCRIPTION OF DRAWINGS

FIG. 1 is a side view of a balloon catheter according to one or more embodiments of the present invention.

FIG. 2 is a cross-sectional view of the balloon catheter taken along line II-II of FIG. 1.

FIG. 3 is a perspective view of a balloon of the balloon catheter illustrated in FIG. 1.

FIG. 4 is a longitudinal sectional view of the balloon according to one or more embodiments of the present invention.

FIG. 5 is a side view of the balloon according to one or more embodiments of the present invention in a deflated state.

FIG. 6 is a cross-sectional view of the balloon taken along line VI-VI of FIG. 5.

FIG. 7 is a cross-sectional view of the balloon taken along line VII-VII of FIG. 5.

FIG. 8 is a perspective view of a balloon according to one or more embodiments of the present invention.

FIG. 9 is a side view of the balloon illustrated in FIG. 8 in a deflated state.

FIG. 10 is a cross-sectional view of the balloon taken along line X-X of FIG. 9.

FIG. 11 is a perspective view of a balloon according to one or more embodiments of the present invention.

FIG. 12 is a longitudinal sectional view of a balloon according to one or more embodiments of the present invention.

FIG. 13 is a side view of the balloon illustrated in FIG. 11 in a deflated state.

FIG. 14 is a cross-sectional view of the balloon taken along line XIV-XIV of FIG. 13.

FIG. 15 is a longitudinal sectional view of a balloon according to one or more embodiments of the present invention.

FIG. 16 is a longitudinal sectional view of a balloon according to one or more embodiments of the present invention.

### DETAILED DESCRIPTION

Hereinafter, one or more embodiments of the present invention will be described on the basis of embodiments, but the present invention is not limited to the following embodiments, and it is of course possible to carry out the present invention by adding appropriate modifications within a range that can be adapted to the gist described above and below, and all of them are included in the technical scope of the present invention. In each drawing, hatching, the reference signs of components, and the like may sometimes be omitted for convenience. In such cases, reference may be made to the specification or the other drawings.

Additionally, the dimensions of various components in the drawings may differ from the actual dimensions as priority is given to facilitating understanding of the features of one or more embodiments of the present invention.

### 1. Balloon for Balloon Catheter

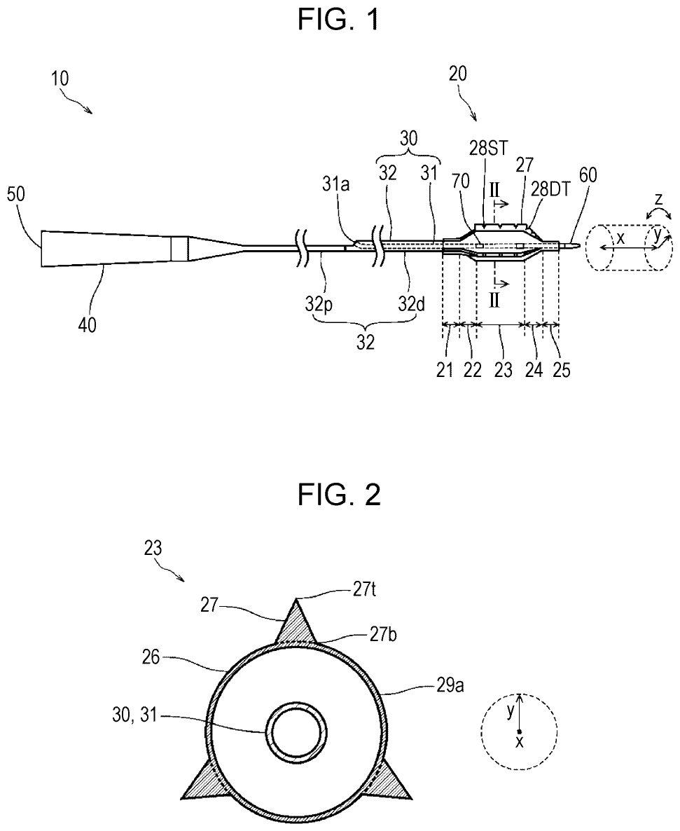

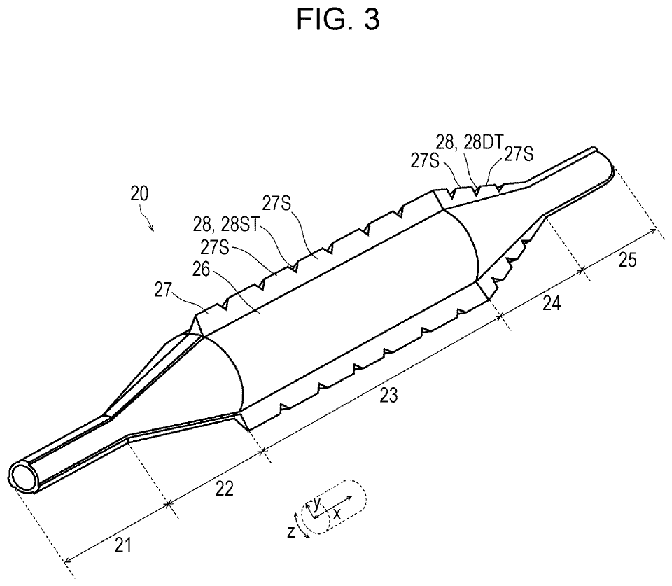

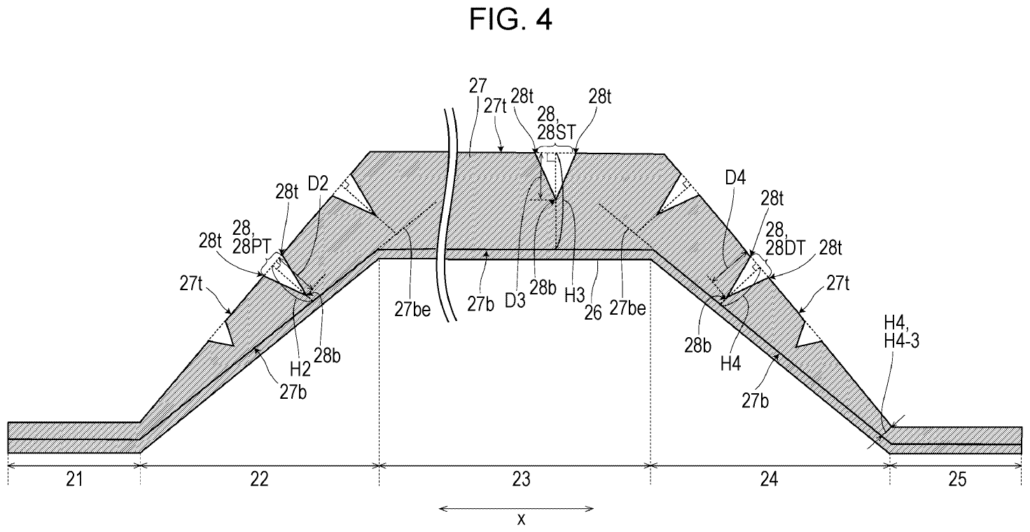

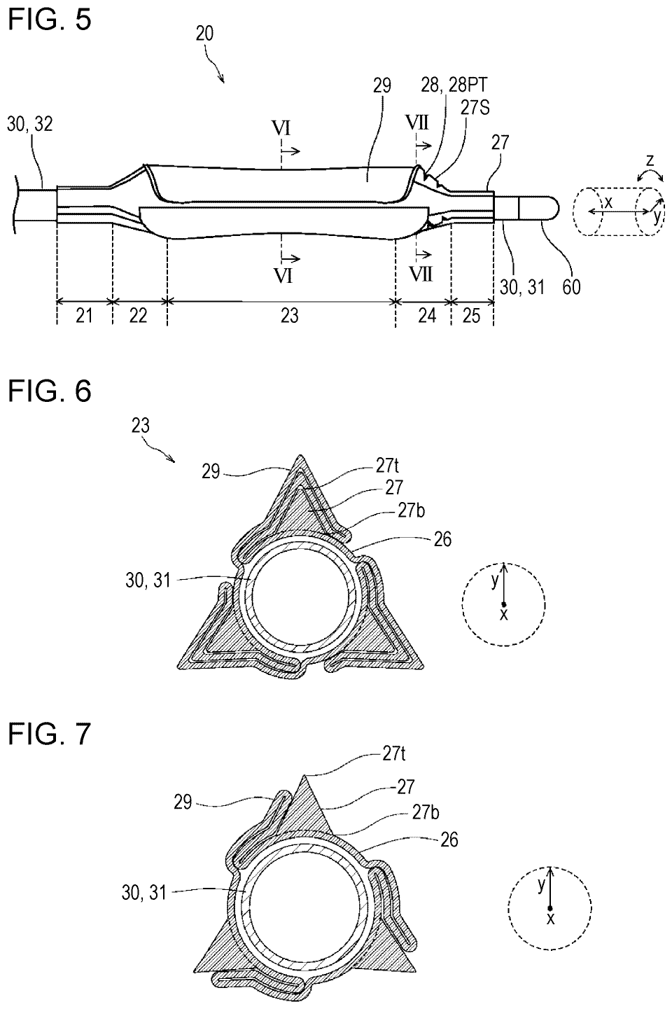

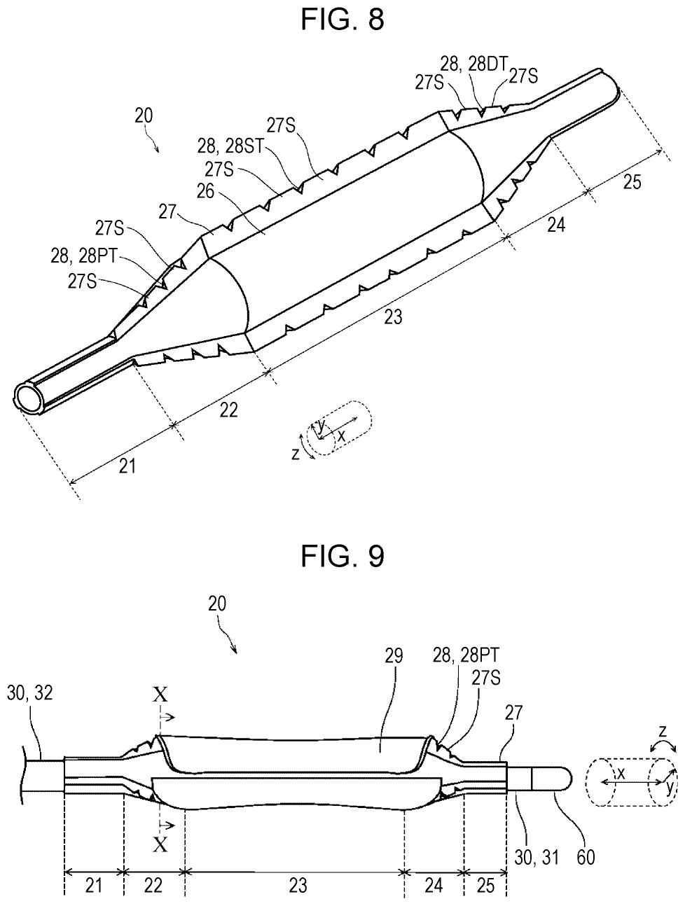

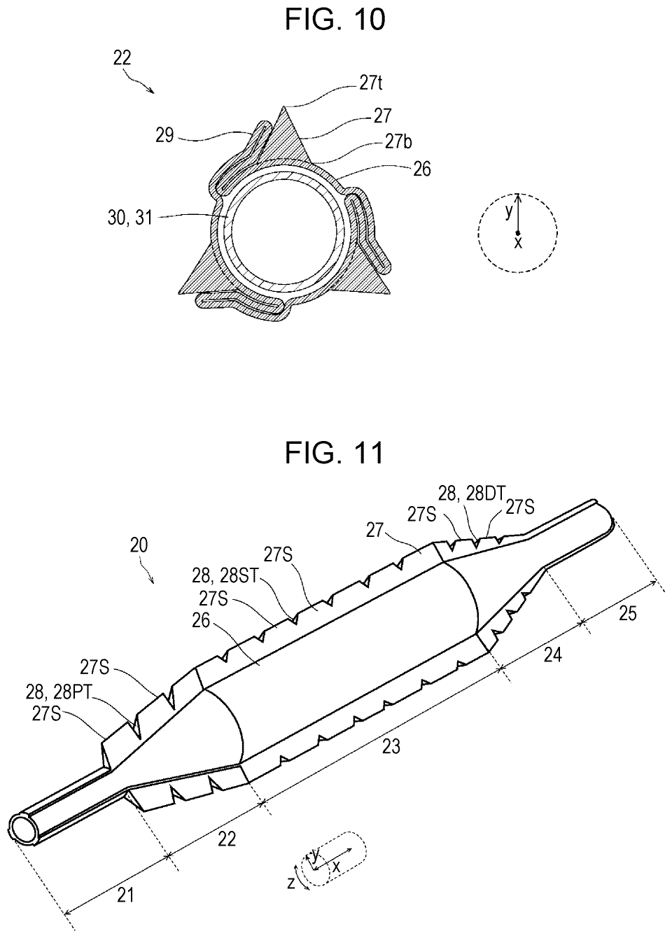

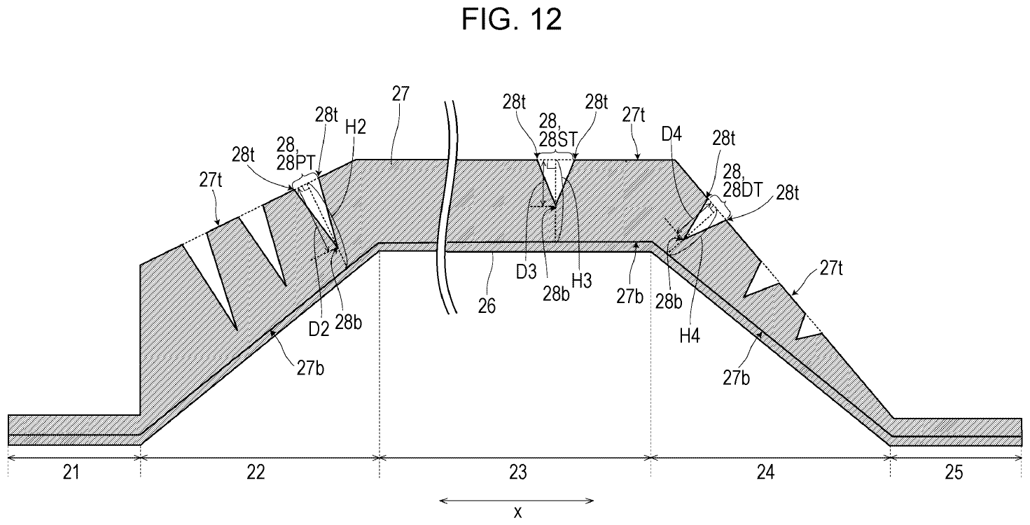

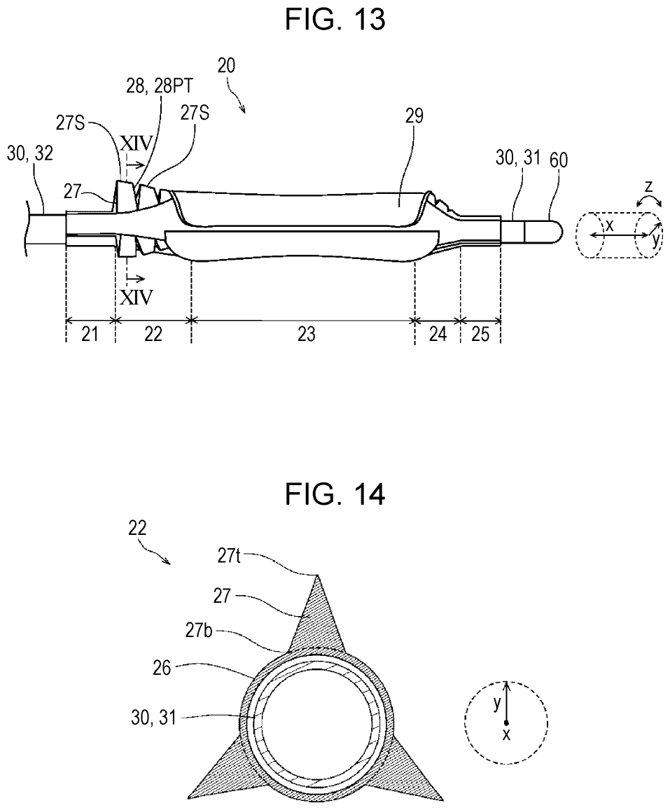

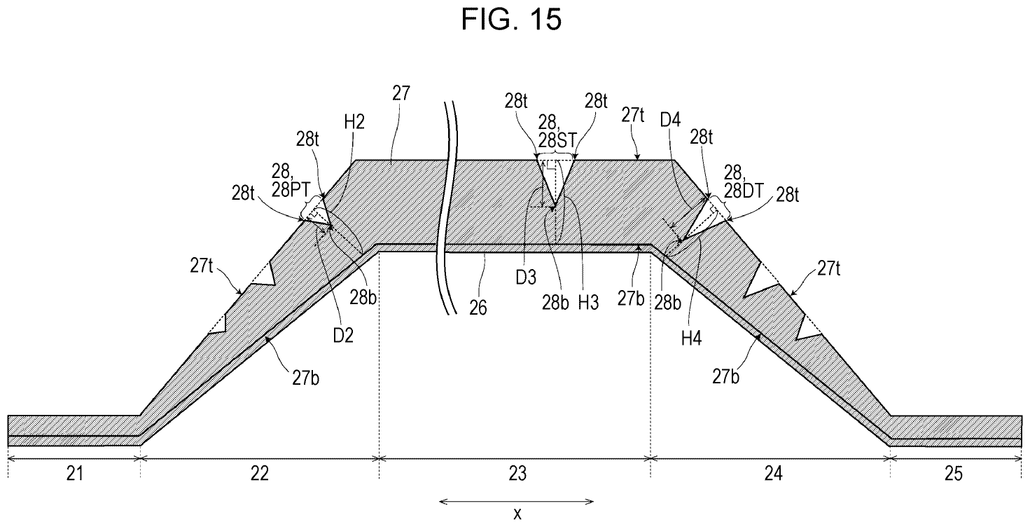

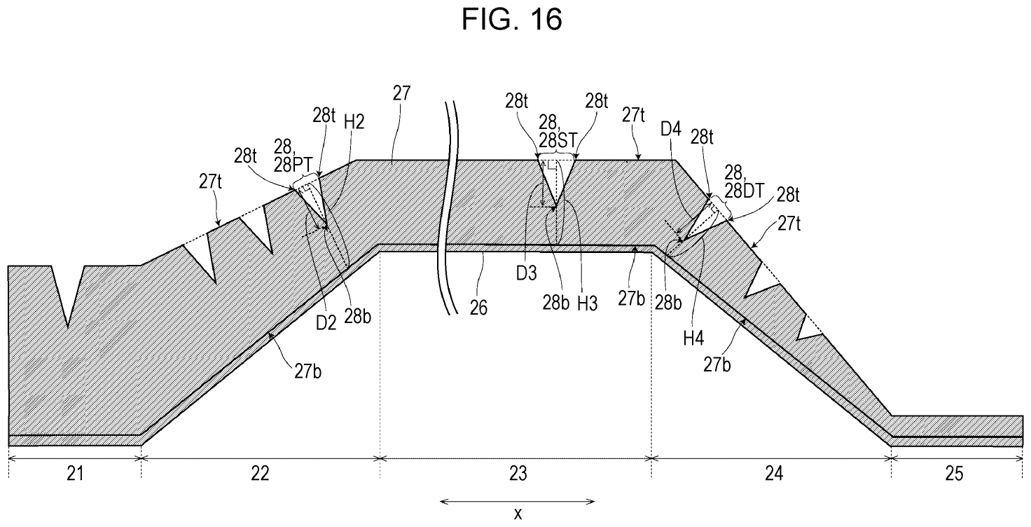

A balloon for a balloon catheter according to one or more embodiments of the present invention will be described with reference to FIGS. 1 to 16. FIG. 1 is a side view of a balloon catheter according to one or more embodiments of the present invention. FIG. 2 is a cross-sectional view of the balloon catheter taken along line II-II of FIG. 1 and illustrates a cross section perpendicular to the longitudinal axis direction of a portion of a straight tube portion of a balloon where a notch is not disposed in the protruding portion. FIG. 3 is a perspective view of the balloon of the balloon catheter illustrated in FIG. 1. FIGS. 2 and 3 illustrate the balloon in an inflated state. FIG. 4 is a cross-sectional view in the longitudinal axis direction of the balloon according to one or more embodiments of the present invention and illustrates, in an enlarged manner, a portion in which the protruding portion is disposed. FIG. 5 is a side view of the balloon according to one or more embodiments of the present invention in a deflated state. FIG. 6 is a cross-sectional view of the balloon taken along line VI-VI of FIG. 5 and illustrates a configuration in which a wing is formed in the straight tube portion and in which the protruding portion of the straight tube portion is covered with the wing while the balloon is in the deflated state. FIG. 7 is a cross-sectional view of the balloon taken along line VII-VII of FIG. 5 and illustrates a configuration in which the wing is formed in a distal-side tapered portion and at least a part of the protruding portion in the distal-side tapered portion is exposed beyond the wing while the balloon is in the deflated state. FIG. 8 is a perspective view of a balloon according to one or more embodiments of the present invention. FIG. 8 illustrates the balloon in an inflated state. FIG. 9 is a side view of the balloon illustrated in FIG. 8 in a deflated state. FIG. 10 is a cross-sectional view of the balloon taken along line X-X of FIG. 9 and illustrates a cross section perpendicular to the longitudinal axis direction of a proximal-side tapered portion while the balloon is in the deflated state. FIG. 11 is a perspective view of a balloon according to one or more embodiments of the present invention. FIG. 11 illustrates the balloon in an inflated state. FIG. 12 is a longitudinal sectional view of a balloon according to one or more embodiments of the present invention and illustrates, in an enlarged manner, a portion where a protruding portion is disposed. FIG. 13 is a side view of the balloon illustrated in FIG. 11 in a deflated state. FIG. 14 is a cross-sectional view of the balloon taken along line XIV-XIV of FIG. 13 and illustrates a cross section perpendicular to the longitudinal axis direction of a proximal-side tapered portion while the balloon is in the deflated state. Each of FIG. 15 and FIG. 16 is a longitudinal sectional view of a balloon according to one or more embodiments of the present invention and illustrates, in an enlarged manner, a portion where a protruding portion is disposed.

As illustrated in FIG. 1, a balloon 20 is provided at a distal portion of a balloon catheter 10. The balloon 20 is connected to a distal end portion of a shaft 30, and the balloon 20 can be inflated by introducing a fluid therein through a lumen of the shaft 30, and the balloon 20 can be deflated by discharging the fluid. In order to control the inflation and deflation of the balloon 20, a fluid can be introduced into and discharged from the balloon 20 by using an indeflator (balloon pressurizer). The fluid may be a pressurized fluid that is pressurized by a pump or the like. The balloon catheter 10 will be described in detail in the section “2. Balloon Catheter”.

As illustrated in FIGS. 1 to 3, the balloon 20 has a longitudinal axis direction x, a radial direction y that is a direction connecting a centroid of a circumscribed circle of the balloon 20 and a point on the circumscribed circle in a cross section perpendicular to the longitudinal axis direction x, and a circumferential direction z that is a direction along the circumscribed circle. In the present specification, a direction toward a user's hand in the longitudinal axis direction x is referred to as a proximal side, and a side opposite to the proximal side, that is, a direction toward a treatment target person is referred to as a distal side.

Members and portions other than the balloon 20 each have a longitudinal axis direction, a radial direction, and a circumferential direction, which may be the same as or different from the longitudinal axis direction x, the radial direction y, and the circumferential direction z of the balloon 20, respectively. However, in the present specification, for ease of understanding, each member and portion is described as having the same longitudinal axis direction, radial direction, and circumferential direction as the longitudinal axis direction x, the radial direction y, and the circumferential direction z of the balloon 20.

As illustrated in FIGS. 1 and 3, the balloon 20 for a balloon catheter according to one or more embodiments of the present invention includes a straight tube portion 23, a proximal-side tapered portion 22 located on the proximal side of the straight tube portion 23, a proximal-side sleeve portion 21 located on the proximal side of the proximal-side tapered portion 22, a distal-side tapered portion 24 located on the distal side of the straight tube portion 23, and a distal-side sleeve portion 25 located on the distal side of the distal-side tapered portion 24.

The proximal-side tapered portion 22 and the distal-side tapered portion 24 may be formed such that the diameter of a balloon main body 26 decreases as the distance from the straight tube portion 23 increases. With such a configuration, the straight tube portion 23 of the balloon 20 can have the maximum diameter when the balloon 20 is in an inflated state, and by inflating the balloon 20 at a lesion portion, the straight tube portion 23 can be brought into sufficient contact with the lesion portion to facilitate expansion and cutting of a stenosed site. In addition, since the balloon 20 includes the proximal-side tapered portion 22 and the distal-side tapered portion 24, when the balloon 20 is deflated, the outer diameter of a proximal end portion of the balloon 20 and the outer diameter of a distal end portion of the balloon 20 can be reduced so as to reduce the difference in diameter between the shaft 30 and the balloon 20, so that it becomes easier to insert the balloon 20 into a body cavity, a forceps channel of an endoscope, or a delivery catheter such as a guiding catheter.

Furthermore, as will be described later, when the balloon 20 is deflated, a balloon main body 26 is formed in at least one wing 29. However, in the proximal-side tapered portion 22 and the distal-side tapered portion 24, the diameter of the balloon main body 26 decreases as the distance from the straight tube portion 23 increases, and thus, the length of the wing 29, which is formed, in the circumferential direction z decreases. Therefore, when the balloon 20 is deflated and the wing 29 is wound around the shaft 30, in the proximal-side tapered portion 22 and the distal-side tapered portion 24, at least a part of at least one protruding portion 27 can be exposed beyond the wing 29, and the stenosed site can be cut by the exposed portion of the protruding portion 27 even when the balloon 20 is deflated.

The proximal-side sleeve portion 21 and the distal-side sleeve portion 25 may be portions that do not inflate even when the balloon 20 is in the inflated state. Accordingly, at least a part of the proximal-side sleeve portion 21 and at least a part of the distal-side sleeve portion 25 can be configured to be stably fixed to the shaft 30. As will be described later, in the case of a configuration in which the shaft 30 includes an inner shaft 31 and an outer shaft 32, at least a part of the proximal-side sleeve portion 21 can be fixed to the outer shaft 32, and at least a part of the distal-side sleeve portion 25 can be fixed to the inner shaft 31.

As illustrated in FIGS. 2 and 3, the balloon 20 includes the balloon main body 26 having an outer surface and an inner surface, and the protruding portion 27 protruding outward in the radial direction y from the outer surface of the balloon main body 26 and extending in the longitudinal axis direction x. The balloon main body 26 is a portion that defines the basic shape of the balloon 20, and the outer shape thereof may be a cylindrical shape in the straight tube portion 23, a truncated cone shape in a tapered portion, and a cylindrical shape having a smaller diameter than the straight tube portion 23 in a sleeve portion. The protruding portion 27 can be rephrased as a portion formed thicker than a portion where the protruding portion 27 is not provided, that is, a portion where the balloon main body 26 is exposed.

The thickness of the balloon 20 in the protruding portion 27 may be, for example, 1.2 times or more, 1.5 times or more, 1.8 times or more, 2.0 times or more, or 2.5 times or more the thickness of the portion of the balloon 20 where the protruding portion 27 is not provided. The upper limit of the thickness of the protruding portion 27 of the balloon 20 is not particularly limited, and may be, for example, 30 times or less, 20 times or less, or 10 times or less the thickness of the portion of the balloon 20 where the protruding portion 27 is not provided.

It is preferable that irregularities not be formed on a portion of the balloon 20 in which the protruding portion 27 is not provided, and it is preferable that the thickness of the portion of the balloon 20 in which the protruding portion 27 is not provided, that is, the thickness of the balloon main body 26 be uniform in the longitudinal axis direction x and the circumferential direction z. Here, the term “irregularities” does not include surface roughness which is inevitably formed in manufacturing. Thus, the balloon 20 can be easily inflated uniformly, and the scoring function of the protruding portion 27 can be easily exerted as desired. However, the thickness of the balloon main body 26 in the straight tube portion 23, the thickness of the balloon main body 26 in the tapered portion, and the thickness of the balloon main body 26 in the sleeve portion may be different from each other, or the thickness of the balloon main body 26 in the straight tube portion 23 may be the thinnest. This makes it possible to provide the balloon 20 with improved flexibility.

As illustrated in FIG. 2, the protruding portion 27 has a top portion 27t and a base portion 27b. The top portion 27t is a portion including an outer end of the protruding portion 27 in the radial direction y, and the base portion 27b is a portion including a boundary with the balloon main body 26, that is, an inner end of the protruding portion 27 in the radial direction y. The top portion 27t does not necessarily have a corner as illustrated in FIG. 2, and may have any shape. Regardless of the shape of the top portion 27t, the outer end of the protruding portion 27 in the radial direction y can be uniquely defined, and a portion including the outer end can be defined as the top portion 27t.

Since the balloon 20 has the protruding portion 27, a scoring function can be imparted to the balloon 20, and by bringing the protruding portion 27 into contact with a lumen wall of a stenosed site and making the protruding portion 27 bite into the lumen wall, a crack can be formed even in a calcified lumen wall of the stenosed site. Therefore, it is possible to expand a stenosed site while suppressing the dissection of a vascular intima. In addition, since the protruding portion 27 can improve the rigidity of the balloon 20 due to the thickness and the rigidity thereof, it is also possible to increase the pressure resistance of the balloon 20 and to suppress excessive inflation of the balloon 20 during pressurization.

As illustrated in FIG. 3, the protruding portion 27 is provided on the outer surface of the balloon main body 26 so as to extend in a ridge-like manner in the longitudinal axis direction x. Note that notches 28, which will be described later, are formed in the protruding portion 27, and the protruding portion 27 extending in a ridge shape in the longitudinal axis direction x may be interrupted by the notches 28. Even in this case, when the protruding portion 27 extends on both the distal and proximal sides of each of the notches 28 in the longitudinal axis direction x, it can be said that the single protruding portion 27 extends in the longitudinal axis direction x.

As illustrated in FIGS. 2 and 3, a plurality of protruding portions 27 may be provided in the circumferential direction z, or, although not illustrated, only one protruding portion 27 may be provided in the circumferential direction z. The number of the protruding portions 27 in the circumferential direction z may be 1 or more, 2 or more, 3 or more, 4 or more, or 6 or more, and may be 20 or less, 15 or less, or 10 or less. In the case where a plurality of protruding portions 27 are provided in the circumferential direction z, the plurality of protruding portions 27 may be separated from each other in the circumferential direction z, and may be arranged at substantially equal intervals in the circumferential direction z. The distance between the protruding portions 27 separated from each other may be longer than the maximum length of the protruding portions 27 in the circumferential direction z.

The protruding portion 27 may be provided in the straight tube portion 23 and the distal-side tapered portion 24, and a range in which the protruding portion 27 is disposed in the longitudinal axis direction x may be a partial section or the entire section of the straight tube portion 23, and may be a partial section or the entire section of the distal-side tapered portion 24. The protruding portion 27 may be disposed in the proximal-side tapered portion 22, and a range in which the protruding portion 27 is disposed in the longitudinal axis direction x may be a partial section or the entire section of the proximal-side tapered portion 22. Further, the protruding portion 27 may be provided in the proximal-side sleeve portion 21 and/or the distal-side sleeve portion 25, and a range in which the protruding portion 27 is disposed in the longitudinal axis direction x may be a partial section or the entire section of the proximal-side sleeve portion 21 and/or the distal-side sleeve portion 25. Here, it is assumed that the range in which the protruding portion 27 is disposed in the longitudinal axis direction x includes portions (described later) of the protruding portion 27 that are separated by the notches 28. In the case where a portion of the balloon main body 26 is exposed by the notches 28, the length of the exposed portion of the balloon main body 26 in the longitudinal axis direction x may be shorter than the maximum length of protruding portion segments 27S (described later) in the longitudinal axis direction x. In this configuration, the single protruding portion 27 extends in the longitudinal axis direction x with the notches 28. Alternatively, the protruding portion 27 may be provided in a part of the proximal-side tapered portion 22 and a part of the straight tube portion 23 in the longitudinal axis direction x, and by making the length of the exposed portion in the longitudinal axis direction x longer than the maximum length of the protruding portion segments 27S in the longitudinal axis direction x, a plurality of protruding portions 27 may be arranged in the longitudinal axis direction x.

The cross-sectional shape of the protruding portion 27 in a cross section perpendicular to the longitudinal axis direction x may be any shape, and may be, for example, a triangle, a quadrangle, a polygon, a semicircle, a part of a circle, a substantially circular shape, a fan shape, a wedge shape, a convex-character shape, a spindle shape, or a combination thereof. Note that the terms “triangle”, “quadrangle”, and “polygon” include not only those with clearly defined vertices and straight sides, but also so-called rounded-corner polygons with rounded corners, and shapes in which at least a part of side portions is curved. Alternatively, the cross-sectional shape of the protruding portion 27 may be an irregular shape having irregularities, chips, or the like. For example, the protruding portion 27 may have a different cross-sectional shape depending on the position in the longitudinal axis direction x. In such a case, for example, although portions of the protruding portion 27 where the notches 28 are not provided have a substantially triangular cross-sectional shape, portions of the protruding portion 27 where the notches 28 are provided may have a substantially trapezoidal cross-sectional shape.

As illustrated in FIG. 3, the protruding portion 27 in the straight tube portion 23 has an ST-portion notch 28ST, and the protruding portion 27 in the distal-side tapered portion 24 has a DT-portion notch 28DT. The ST-portion notch 28ST is a notch provided in the straight tube portion 23, and the DT-portion notch 28DT is a notch provided in the distal-side tapered portion 24. Hereinafter, the notches may be collectively referred to as the notches 28 regardless of the locations where they are provided.

Each of the notches 28 can be provided such that a recess is formed in the top portion 27t of the protruding portion 27 extending in the longitudinal axial direction x. In this case, each of the notches 28 may be formed by cutting off a part of the top portion 27t of the protruding portion 27. As illustrated in FIG. 4, each of the notches 28 has a bottom portion 28b (described later) located at the deepest position in the depth direction of the notch 28, and a top portion 28t that is a boundary with the top portion 27t of the protruding portion 27. Since each of the notches 28 is provided such that a recess is formed in the top portion 27t of the protruding portion 27, it is preferable that each of the notches 28 have two top portions 28t in a cross section in the longitudinal axis direction x passing through the top portion 27t of the protruding portion 27.

Both a depth D3 of the ST-portion notch 28ST and a depth D4 of the DT-portion notch 28DT may be 50 μm or more. Both the depth D3 and the depth D4 may be 70 μm or more or 100 μm or more. When the lower limit of the depth of each of the notches 28 is equal to or greater than the above, the protruding portion 27 can be divided into a plurality of protruding portion segments 27S in the longitudinal axis direction x. That is, by providing the straight tube portion 23 with the ST-portion notch 28ST, the protruding portion segments 27S can be arranged at both ends of the ST-portion notch 28ST in the longitudinal axis direction x, and by providing the distal-side tapered portion 24 with the DT-portion notch 28DT, the protruding portion segments 27S can be arranged at both ends of the DT-portion notch 28DT in the longitudinal axis direction x. In the longitudinal axis direction x, the top portions 28t of the notches 28 are located at the end portions of the respective protruding portion segments 27S.

The upper limit of the depth of each of the notches 28 is the height of the protruding portion 27 at the position where the notch 28 is provided. A method of determining the height of the protruding portion 27 will be described later. The depth D3 of the ST-portion notch 28ST may be 500 μm or less, 400 μm or less, 300 μm or less, or 200 μm or less. The depth D4 of the DT-portion notch 28DT may be 500 μm or less, 400 μm or less, 300 μm or less, or 200 μm or less.

As illustrated in FIGS. 3 and 4, the depth D3 of the ST-portion notch 28ST, a height H3 of the protruding portion 27 in the straight tube portion 23, the depth D4 of the DT-portion notch 28DT, and a height H4 of the protruding portion 27 in the distal-side tapered portion 24 satisfy a relationship of D3/H3≤D4/H4.

As illustrated in FIG. 5, the balloon 20 can be brought into a deflated state before the fluid is introduced into the lumen thereof or after the fluid once introduced has been discharged. When the balloon 20 is in the inflated state, as illustrated in FIG. 2, the balloon main body 26 in the straight tube portion 23 has a cylindrical shape, but when the balloon 20 is in the deflated state, as illustrated in FIGS. 5 to 7, the wing 29 is formed on the balloon 20, and as a result of the wing 29 being folded, the outer diameter of the balloon 20 can be reduced such that the inner wall of the balloon main body 26 is brought closer to the shaft 30. That is, as illustrated in FIG. 2, it can be said that the balloon 20 in the inflated state has a wing forming portion 29a, and the longer the wing forming portion 29a is in the circumferential direction z, the longer the wing 29 can be formed. As illustrated in FIG. 6, since the straight tube portion 23 is a portion having the maximum diameter when the balloon 20 is inflated, the length of the wing 29 in the straight tube portion 23 is long, and the protruding portion 27 in the straight tube portion 23 can be covered with the wing 29 in the deflated state.

On the other hand, as illustrated in FIG. 7, the wing 29 is short in the distal-side tapered portion 24 in which the outer diameter of the balloon main body 26 gradually decreases from the straight tube portion 23 when the balloon main body 26 is inflated, and thus, at least a part of the protruding portion 27 in the distal-side tapered portion 24, in particular, the top portion 27t of the protruding portion 27 can be exposed beyond the wing 29 even in the deflated state. As illustrated in FIG. 5, on the more distal side of the distal-side tapered portion 24, the protruding portion 27 can be exposed almost without being affected by the wing 29. As a result, the protruding portion 27 in the distal-side tapered portion 24 can contribute to cutting of a stenosed site in a state where the wing 29 is folded when the balloon 20 is deflated.

In this case, since the value of the ratio D4/H4 of the depth D4 of the DT-portion notch 28DT of the protruding portion 27 in the distal-side tapered portion 24 to the height H4 of the protruding portion 27 in the distal-side tapered portion 24 is equal to or larger than the value of the ratio D3/H3 of the depth D3 of the ST-portion notch 28ST of the protruding portion 27 in the straight tube portion 23 to the height H3 of the protruding portion 27 in the straight tube portion 23, each protruding portion segment 27S divided by the DT-portion notch 28DT in the distal-side tapered portion 24 can easily act on the stenosed site, and it is possible to enhance the effect of cutting the stenosed site with edges that are formed at boundary portions between the protruding portion segments 27S and the DT-portion notch 28DT, that is, formed at the top portions 28t of the notch 28, and the effect of scraping the stenosed site. Therefore, by performing an operation such as advancing the balloon 20 when the balloon 20 is deflated (inching the balloon 20 forward), it is possible to expand the stenosed site by the protruding portion 27 in the distal-side tapered portion 24.

The ratio D3/H3 of the depth D3 of the ST-portion notch 28ST of the protruding portion 27 to the height H3 of the protruding portion 27 in the straight tube portion 23 is equal to or smaller than the ratio D4/H4 of the depth D4 of the DT-portion notch 28DT of the protruding portion 27 in the distal-side tapered portion 24 to the height H4 of the protruding portion 27 in the distal-side tapered portion 24. As a result, the rigidity of the protruding portion 27 in the straight tube portion 23 can be improved, and the function of expanding the stenosed site by the protruding portion 27 in the straight tube portion 23 can be improved in the state where the protruding portion 27 in the straight tube portion 23 is exposed beyond the wing 29 when the balloon 20 is inflated. Furthermore, when the balloon 20 is deflated and the wing 29 is folded, the wing 29 can be prevented from being caught by the ST-portion notch 28ST in the straight tube portion 23. As a result, since the wing 29 can be easily folded to reduce the outer diameter of the balloon 20, it is possible to improve the maneuverability of the balloon 20 when the balloon 20 is deflated and removed from the body or when the balloon 20 is reinserted into another treatment site.

As illustrated in FIG. 4, regarding the depth of each of the notches 28, in a cross section in the longitudinal axis direction x passing through the top portion 27t of the protruding portion 27, when a perpendicular line is drawn from an imaginary straight line connecting the two top portions 28t of the notch 28 toward the bottom portion 28b of the notch 28, the depth of the notch 28 can be determined to be the distance between the point where the perpendicular line intersects the imaginary straight line and the point where the perpendicular line intersects the bottom portion 28b. In the case illustrated in FIG. 4, the imaginary straight line connecting the top portions 28t of the ST-portion notch 28ST is parallel to the longitudinal axis direction x, and the depth D3 is determined as the distance between the imaginary straight line and the bottom portion 28b of the ST-portion notch 28ST in the radial direction y. In the distal-side tapered portion 24, an imaginary straight line connecting the top portions 28t of the DT-portion notch 28DT is not parallel to the longitudinal axis direction x, but the depth D4 can be determined by drawing a perpendicular line from the imaginary straight line toward the bottom portion 28b of the DT-portion notch 28DT. The cross section in the longitudinal axis direction x passing through the top portion 27t of the protruding portion 27 is a cross section passing through the centroid of the balloon main body 26 in a cross section perpendicular to the longitudinal axis direction x, that is, the central axis of the balloon main body 26. In the present specification, the same applies to other portions described as “a cross section in the longitudinal axis direction x passing through the top portion 27t of the protruding portion 27”.

The shape of each of the notches 28 is not particularly limited. In a cross section in the longitudinal axis direction x passing through the top portion 27t of the protruding portion 27, the shape of each of the notches 28 may be a V-shape, a U-shape, a shape obtained by removing one side of a rectangle, a shape obtained by combining these shapes, or the like. In any shape, since the bottom portion 28b located at the deepest position in the depth direction can be specified, the depth can be determined by the above definition. The bottom portion 28b of each of the notches 28 may or may not reach the base portion 27b of the protruding portion 27. The balloon main body 26 may be exposed at the bottom of at least one of the notches 28 by the bottom portion 28b of the notch 28 reaching the base portion 27b of the protruding portion 27. Even in the case where the balloon main body 26 is exposed at the bottom of at least one of the notches 28, if the length of the exposed portion in the longitudinal axis direction x is shorter than the maximum length of a respective one of the protruding portion segments 27S in the longitudinal axis direction x, the exposed portion is regarded as a part of the notch 28 rather than a portion where the protruding portion 27 is not present.

As illustrated in FIG. 4, the height H3 of the protruding portion 27 in the straight tube portion 23 is measured at a portion where the notch 28 (ST-portion notch 28ST) is provided. In a cross section in the longitudinal axis direction x passing through the top portion 27t of the protruding portion 27, when a perpendicular line is drawn from an imaginary straight line connecting the two top portions 28t of the notch 28 (ST-portion notch 28ST) toward the bottom portion 28b of the notch 28 (ST-portion notch 28ST), the height H3 of the protruding portion 27 in the straight tube portion 23 can be determined to be the distance between the point where the perpendicular line intersects the imaginary straight line and the point where the perpendicular line intersects the base portion 27b of the protruding portion 27. Similarly, the height H4 of the protruding portion 27 in the distal-side tapered portion 24 is measured at a portion where the notch 28 (DT-portion notch 28DT) is provided. In a cross section in the longitudinal axis direction x passing through the top portion 27t of the protruding portion 27, when a perpendicular line is drawn from an imaginary straight line connecting the two top portions 28t of the notch 28 (DT-portion notch 28DT) toward the bottom portion 28b of the notch 28 (DT-portion notch 28DT), the height H4 of the protruding portion 27 in the distal-side tapered portion 24 can be determined to be the distance between the point where the perpendicular line intersects the imaginary straight line and the point where the perpendicular line intersects the base portion 27b of the protruding portion 27. Even in the case where the protruding portion 27 is disposed in another portion, which is, for example, the proximal-side tapered portion 22, the height of the protruding portion 27 can be determined by a similar method as long as the notch 28 is provided.

For example, in the case where the protruding portion 27 is provided in the proximal-side sleeve portion 21, the distal-side sleeve portion 25, or the like and where the notches 28 are not provided, when a perpendicular line is drawn, in a cross section in the longitudinal axis direction x passing through the top portion 27t of the protruding portion 27, from an imaginary straight line connecting a proximal end and a distal end of the top portion 27t of the protruding portion 27 in the corresponding portion (e.g., in the case of the proximal-side sleeve portion 21, an imaginary straight line connecting the proximal end and the distal end of the top portion 27t of the protruding portion 27 in the proximal-side sleeve portion 21, and in the case of the distal-side sleeve portion 25, an imaginary straight line connecting the proximal end and the distal end of the top portion 27t of the protruding portion 27 in the distal-side sleeve portion 25), the height of the protruding portion 27 in each of these portions can be determined to be the distance between the point where the perpendicular line intersects the top portion 27t of the protruding portion 27 and the point where the perpendicular line intersects the base portion 27b of the protruding portion 27. When the height of the protruding portion 27 in each portion is not constant along the longitudinal axis direction x, the average of the heights of the protruding portion 27 at three different points spaced apart from each other in the longitudinal axis direction x, that is, for example, three points including a proximal end, a middle point, and a distal end in each portion can be determined to be the height of the protruding portion 27 of the portion. In the case where the perpendicular line does not intersect the base portion 27b of the protruding portion 27, as will be described later, the base portion 27b of the protruding portion 27 is extrapolated, and a point at which the perpendicular line intersects an extrapolated line 27be can be used as a reference for the height.

In FIG. 4, although the outer edge of the protruding portion 27 is formed linearly in the cross section in the longitudinal axis direction x passing through the top portion 27t of the protruding portion 27, the shape of the protruding portion 27 is not limited thereto, and the outer edge of the protruding portion 27 in the cross section in the longitudinal axis direction x passing through the top portion 27t of the protruding portion 27, that is, the top portion 27t, may have any shape such as a curved shape or a shape combining a straight line and a curve from the proximal end to the distal end of each of the straight tube portion 23 and the distal-side tapered portion 24.

When the straight tube portion 23 or the distal-side tapered portion 24 is provided with two or more of the notches 28, the height of the protruding portion 27 is measured at each of the portions where the notches 28 are provided, and the average of the measured heights is determined. For example, when five ST-portion notches 28ST are provided in the straight tube portion 23, the height of the protruding portion 27 is measured at five positions where the ST-portion notches 28ST are provided, and the average of the measured five heights is set as the height H3 of the protruding portion 27 in the straight tube portion 23. Further, for example, in the case where three DT-portion notches 28DT are provided in the distal-side tapered portion 24, the height of the protruding portion 27 is measured at three positions where the DT-portion notches 28DT are provided, and the average of the measured three heights is set as the height H4 of the protruding portion 27 in the distal-side tapered portion 24. In the above-described case, the number of the ST-portion notches 28ST provided in the straight tube portion 23 is five, and the number of the DT-portion notches 28DT provided in the distal-side tapered portion 24 is three. However, the number of the notches 28 is not limited thereto. In the case where the height of the protruding portion 27 is not constant along the longitudinal axis direction x as in the distal-side tapered portion 24, when a perpendicular line is drawn from an imaginary straight line connecting the two top portions 28t of one of the notches 28 toward the bottom portion 28b of the notch 28 to determine the height of the protruding portion 27, the perpendicular line may not intersect the base portion 27b of the protruding portion 27. In such a case, an imaginary straight line connecting the proximal and distal ends of the base portion 27b of the protruding portion 27 in the distal-side tapered portion 24 is extrapolated, and a point at which the perpendicular line intersects the extrapolated line 27be can be used as a reference for the height. In the case where a perpendicular line does not intersect the base portion 27b of the protruding portion 27 also in portions other than the distal-side tapered portion 24, the base portion 27b of the protruding portion 27 is extrapolated, and a point at which the perpendicular line intersects the extrapolated line 27be can be used as a reference for the height.

In the case where the straight tube portion 23 and/or the distal-side tapered portion 24 is provided with two or more of the notches 28, it is only necessary that any one pair of the notches 28 satisfy the relationship of D3/H3≤D4/H4, where one notch is from the straight tube portion 23 and the other is from the distal-side tapered portion 24. Alternatively, all of the plurality of notches 28 may satisfy the relationship of D3/H3≤D4/H4.

Alternatively, it is preferable that the ST-portion notch 28ST of the protruding portion 27 in the straight tube portion 23 include a plurality of ST-portion notches 28ST and that the depth D3 be an average of the depths of the plurality of ST-portion notches 28ST. Even when comparing the average of the depths and the average of the heights, the above-described effects can be achieved by satisfying the relationship of D3/H3≤D4/H4.

It is preferable that the DT-portion notch 28DT of the protruding portion 27 in the distal-side tapered portion 24 include a plurality of DT-portion notches 28DT and that the depth D4 be an average of the depths of the plurality of DT-portion notches 28DT. Even when comparing the average of the depths and the average of the heights, the above-described effects can be achieved by satisfying the relationship of D3/H3≤D4/H4.

The relationship of D3/H3≤D4/H4 may be satisfied by the bottom portion 28b of the ST-portion notch 28ST not reaching the base portion 27b of the protruding portion 27 while the bottom portion 28b of the DT-portion notch 28DT reaching the base portion 27b of the protruding portion 27.

Alternatively, the relationship of D3/H3≤D4/H4 may be satisfied in a state where both the bottom portion 28b of the ST-portion notch 28ST and the bottom portion 28b of the DT-portion notch 28DT do not reach the base portion 27b of the protruding portion 27. Alternatively, the relationship of D3/H3≤D4/H4 may be satisfied in a state where both the bottom portion 28b of the ST-portion notch 28ST and the bottom portion 28b of the DT-portion notch 28DT reach the base portion 27b of the protruding portion 27.

In the longitudinal axis direction x, the width of one of the notches 28, that is, the distance between the two top portions 28t of the notch 28, may be smaller than the maximum length of the protruding portion segments 27S in the longitudinal axis direction x. The maximum length of the protruding portion segments 27S in the longitudinal axis direction x is the length of the longest of the plurality of protruding portion segments 27S divided and formed by the notch 28. Thus, the length of the protruding portion segment 27S in the longitudinal axis direction x, which can contribute to cutting of a stenosed site, can be relatively increased to ensure the function of expanding the stenosed site. In addition, the rigidity of the protruding portion 27 can be set to a predetermined value or more.

As illustrated in FIG. 3, the most proximal one of the protruding portion segments 27S in the straight tube portion 23 may be arranged continuously with the most distal one of the protruding portion segments 27S in the proximal-side tapered portion 22. As a result, the protruding portion 27 can be formed continuously from the straight tube portion 23 to the proximal-side tapered portion 22, and it becomes easy to improve the rigidity of the protruding portion 27 to improve the function of expanding a stenosed site and to improve the rigidity of the balloon 20.

As illustrated in FIGS. 3 and 4, the height H3 of the protruding portion 27 in the straight tube portion 23 and the height H4 of the protruding portion 27 in the distal-side tapered portion 24 may satisfy a relationship of H3≥H4. The height H3 of the protruding portion 27 in the straight tube portion 23 and the height H4 of the protruding portion 27 in the distal-side tapered portion 24 may be the same, that is, H3=H4, but the height H3 of the protruding portion 27 in the straight tube portion 23 may be higher than the height H4 of the protruding portion 27 in the distal-side tapered portion 24, that is, H3≥H4. As a result, the outer diameter of the balloon 20 at the distal portion can be kept small, and thus, the maneuverability of the balloon 20 can be improved.

Although not illustrated in the drawings, the balloon 20 may have an inner protruding portion that protrudes inward in the radial direction y and extends in the longitudinal axis direction x on the inner surface of the balloon main body 26. The inner protruding portion may be disposed at the same position as the protruding portion 27 in the longitudinal axis direction x or the circumferential direction z, and the inner protruding portion and the protruding portion 27 may be integrally formed. Accordingly, a part of the balloon 20 may be formed to have a large thickness.

As illustrated in FIGS. 3 to 5, it is preferable that the height of the protruding portion 27 in the distal-side tapered portion 24 gradually decrease from the proximal side to the distal side in the longitudinal axis direction x. This makes it easy to improve the maneuverability of the balloon 20. In this case, it is preferable that the depths D4 of the plurality of DT-portion notches 28DT of the protruding portion 27 in the distal-side tapered portion 24 increase toward the proximal side and decrease toward the distal side. This makes it possible to ensure the rigidity of the protruding portion 27 in the distal-side tapered portion 24. In addition, in this case, it is also a preferable aspect that an inner protruding portion is formed in the distal-side tapered portion 24. As a result, the rigidity of a portion of the balloon 20 on the distal side can be improved, and kinking and excessive inflation during pressurization can be suppressed.

The distal-side sleeve portion 25 may or may not be provided with the protruding portion 27. In the case where the distal-side sleeve portion 25 is provided with the protruding portion 27, the height of the protruding portion 27 may be higher or lower than the height H3 of the protruding portion 27 in the straight tube portion 23, but it is also a preferable aspect that the distal-side sleeve portion 25 is provided with the protruding portion 27 having a low height such as that illustrated in FIG. 3. By not providing the distal-side sleeve portion 25 with the protruding portion 27, or even by providing the distal-side sleeve portion 25 with the protruding portion 27 having a low height, the maneuverability of the balloon 20 can be improved. In this case, the inner protruding portion may be provided in the distal-side sleeve portion 25.

Portions of the balloon 20 on the proximal side, that is, the proximal-side tapered portion 22 and the proximal-side sleeve portion 21, may or may not be provided with the protruding portion 27. As illustrated in FIG. 3, each of the proximal-side tapered portion 22 and the proximal-side sleeve portion 21 may be provided with the protruding portion 27 having a low height. This can improve the maneuverability of the balloon 20.

In the case where the proximal-side tapered portion 22 and/or the proximal-side sleeve portion 21 is not provided with the protruding portion 27, an inner protruding portion may be provided. Alternatively, in the case where the proximal-side tapered portion 22 and/or the proximal-side sleeve portion 21 is provided with the protruding portion 27 but the protruding portion 27 has a low height, the inner protruding portion may be provided at the same position as the protruding portion 27 in the longitudinal axis direction x and the circumferential direction z. As a result, the rigidity of a portion of the balloon 20 on the proximal side can be improved, and kinking and excessive inflation during pressurization can be suppressed.

It is preferable that the protruding portion 27 be provided in the proximal-side tapered portion 22, that the protruding portion 27 in the proximal-side tapered portion 22 have a PT-portion notch 28PT, and that a depth D2 of the PT-portion notch 28PT be 50 μm or more. It is preferable that the depth D3 of the ST-portion notch 28ST in the straight tube portion 23, the height H3 of the protruding portion 27 in the straight tube portion 23, the depth D2 of the PT-portion notch 28PT, and the height H2 of the protruding portion 27 in the proximal-side tapered portion 22 satisfy a relationship of D3/H3≤D2/H2.

As illustrated in FIGS. 9 and 10, in the proximal-side tapered portion 22 in which the outer diameter of the balloon main body 26 gradually decreases from the straight tube portion 23 when the balloon main body 26 is inflated, the length of the wing 29 is short, and thus, at least a part of the protruding portion 27 in the proximal-side tapered portion 22, particularly, the top portion 27t of the protruding portion 27 can be exposed beyond the wing 29 even in the deflated state. As illustrated in FIG. 9, on the more proximal side of the proximal-side tapered portion 22, the protruding portion 27 can be exposed almost without being affected by the wing 29. As a result, the protruding portion 27 in the proximal-side tapered portion 22 can contribute to cutting of a stenosed site in a state where the wing 29 is folded when the balloon 20 is deflated.

In this case, since the value of the ratio D2/H2 of the depth D2 of the PT-portion notch 28PT of the protruding portion 27 in the proximal-side tapered portion 22 to the height H2 of the protruding portion 27 in the proximal-side tapered portion 22 is equal to or larger than the value of the ratio D3/H3 of the depth D3 of the ST-portion notch 28ST of the protruding portion 27 in the straight tube portion 23 to the height H3 of the protruding portion 27 in the straight tube portion 23, each protruding portion segment 27S divided by the PT-portion notch 28PT in the proximal-side tapered portion 22 can easily act on the stenosed site, and it is possible to enhance the effect of cutting the stenosed site with edges that are formed at boundary portions between the protruding portion segments 27S and the PT-portion notch 28PT, that is, formed at the top portions 28t of the notch 28, and the effect of scraping the stenosed site. Therefore, by performing an operation such as advancing the balloon 20 when the balloon 20 is deflated (inching the balloon 20 forward) as illustrated in FIG. 9, it is possible to expand the stenosed site by the protruding portion 27 in the proximal-side tapered portion 22.

In the case where the straight tube portion 23 and/or the proximal-side tapered portion 22 is provided with two or more of the notches 28, it is only necessary that any one pair of the notches 28 satisfy the relationship of D3/H3≤D2/H2, where one notch is from the straight tube portion 23 and the other is from the proximal-side tapered portion 22. Alternatively, all of the plurality of notches 28 may satisfy the relationship of D3/H3≤D2/H2.

The depth D2 of the PT-portion notch 28PT may be 70 μm or more, or 100 μm or more. When the lower limit of the depth D2 of the PT-portion notch 28PT is equal to or greater than the above, the protruding portion 27 can be easily divided into the plurality of protruding portion segments 27S in the longitudinal axis direction x. The depth D2 of the PT-portion notch 28PT may be 500 μm or less, 400 μm or less, 300 μm or less, 200 μm or less, or 100 μm or less.

Alternatively, it is preferable that the PT-portion notch 28PT of the protruding portion 27 in the proximal-side tapered portion 22 include a plurality of PT-portion notches 28PT and that the depth D2 be an average of the depths of the plurality of PT-portion notches 28PT. Even when comparing the average of the depths and the average of the heights, the above-described effects can be achieved by satisfying the relationship of D3/H3≤D2/H2.

For example, as illustrated in FIGS. 4, 8, and 9, as a result of the height H3 of the protruding portion 27 in the straight tube portion 23 and the height H2 of the protruding portion 27 in the proximal-side tapered portion 22 satisfying a relationship of H3≥H2, the relationship of D3/H3≤D2/H2 may be satisfied. The height H3 of the protruding portion 27 in the straight tube portion 23 and the height H2 of the protruding portion 27 in the proximal-side tapered portion 22 may be the same, that is, H3=H2, but the height H3 of the protruding portion 27 in the straight tube portion 23 may be higher than the height H2 of the protruding portion 27 in the proximal-side tapered portion 22, that is, H3>H2. As a result, the outer diameter of the balloon 20 at the proximal portion can be kept small, and thus, the maneuverability of the balloon 20 can be improved.

As illustrated in FIGS. 4, 8, and 9, it is preferable that the height of the protruding portion 27 in the proximal-side tapered portion 22 gradually decrease from the distal side to the proximal side in the longitudinal axis direction x. This makes it easier to improve the maneuverability of the balloon 20. In this case, if the protruding portion 27 in the proximal-side tapered portion 22 is provided with the plurality of PT-portion notches 28PT, it is preferable that the depths D2 increase toward the distal side and decrease toward the proximal side. This makes it easy to ensure the rigidity of the protruding portion 27 in the proximal-side tapered portion 22. In this case, it is also a preferable aspect that an inner protruding portion is formed in the proximal-side tapered portion 22. As a result, the rigidity of the portion of the balloon 20 on the proximal side can be further improved, and kinking and excessive inflation during pressurization can be suppressed.

Alternatively, as illustrated in FIGS. 11 to 14, the relationship of D3/H3≤D2/H2 may be satisfied in a state where the height H3 of the protruding portion 27 in the straight tube portion 23 and the height H2 of the protruding portion 27 in the proximal-side tapered portion 22 satisfy a relationship of H3<H2. In this case, as a result of the depth D2 of the PT-portion notch 28PT of the protruding portion 27 in the proximal-side tapered portion 22 being relatively deep, the relationship of D3/H3≤D2/H2 can be satisfied. Thus, in the proximal-side tapered portion 22, each of the protruding portion segments 27S divided by the PT-portion notch 28PT can more easily act on a stenosed site, and the effect of cutting the stenosed site with edges that are formed at the boundary portions between the protruding portion segments 27S and the PT-portion notch 28PT, that is, formed at the top portions 28t of the notch 28 and the effect of scraping the stenosed site can be further improved. Therefore, by performing an operation such as advancing the balloon 20 when the balloon 20 is deflated (inching the balloon 20 forward) as illustrated in FIG. 13, it is possible to more easily expand the stenosed site by the protruding portion 27 in the proximal-side tapered portion 22.

In this case, in the proximal-side tapered portion 22 in which the outer diameter of the balloon main body 26 gradually decreases from the straight tube portion 23 when the balloon main body 26 is inflated, the length of the wing 29 is short, and thus, at least a part of the protruding portion 27 in the proximal-side tapered portion 22, particularly, the top portion 27t of the protruding portion 27 can be exposed beyond the wing 29 even in the deflated state. In particular, as illustrated in FIG. 14, on the more proximal side of the proximal-side tapered portion 22, the protruding portion 27 can be exposed almost without being affected by the wing 29, and the protruding portion 27 can easily contribute to cutting of a stenosed site in a state where the wing 29 is folded when the balloon 20 is deflated.

As illustrated in FIGS. 11 to 13, the height of the protruding portion 27 in the proximal-side tapered portion 22 may gradually increase from the distal side to the proximal side in the longitudinal axis direction x. Accordingly, in combination with the fact that the value of the ratio D2/H2 is relatively large, the protruding portion segments 27S of the protruding portion 27 in the proximal-side tapered portion 22 can effectively act on a stenosed site, so that the function of expanding the stenosed site can be further improved. In addition, even when the value of the ratio D2/H2 is relatively large, it is easy to ensure the rigidity of the protruding portion 27 in the proximal-side tapered portion 22.

Alternatively, as another aspect in which the protruding portion 27 in the proximal-side tapered portion 22 has the PT-portion notch 28PT, an aspect is also preferable in which the depth D2 of the PT-portion notch 28PT is 50 μm or more and in which the depth D3 of the ST-portion notch 28ST in the straight tube portion 23, the height H3 of the protruding portion 27 in the straight tube portion 23, the depth D2 of the PT-portion notch 28PT, and the height H2 of the protruding portion 27 in the proximal-side tapered portion 22 satisfy a relationship of D3/H3>D2/H2.

Since the length of the wing 29 is short in the proximal-side tapered portion 22 in which the outer diameter of the balloon main body 26 gradually decreases from the straight tube portion 23 when the balloon main body 26 is inflated, the protruding portion 27 in the proximal-side tapered portion 22 can contribute to expansion of a stenosed site when the balloon 20 is deflated. In this case, the value of the ratio D2/H2 of the depth D2 of the PT-portion notch 28PT of the protruding portion 27 in the proximal-side tapered portion 22 to the height H2 of the protruding portion 27 in the proximal-side tapered portion 22 is smaller than the value of the ratio D3/H3 of the depth D3 of the ST-portion notch 28ST of the protruding portion 27 in the straight tube portion 23 to the height H3 of the protruding portion 27 in the straight tube portion 23, and thus, the rigidity of the protruding portion 27 of the proximal-side tapered portion 22 can be improved, thereby improving the function of expanding a stenosed site when the balloon 20 is deflated.

In the case where the straight tube portion 23 and/or the proximal-side tapered portion 22 is provided with two or more of the notches 28, it is only necessary that any one pair of the notches 28 satisfy the relationship of D3/H3>D2/H2, where one notch is from the straight tube portion 23 and the other is from the proximal-side tapered portion 22. Alternatively, all of the plurality of notches 28 may satisfy the relationship of D3/H3>D2/H2.

Alternatively, it is preferable that the PT-portion notch 28PT of the protruding portion 27 in the proximal-side tapered portion 22 include a plurality of PT-portion notches 28PT and that the depth D2 be an average of the depths of the plurality of PT-portion notches 28PT. Even when comparing the average of the depths and the average of the heights, the above-described effects can be achieved by satisfying the relationship of D3/H3>D2/H2.

For example, as illustrated in FIG. 15, the relationship of D3/H3>D2/H2 may be satisfied, with the height H3 of the protruding portion 27 in the straight tube portion 23 and the height H2 of the protruding portion 27 in the proximal-side tapered portion 22 satisfying the relationship of H3>H2. The height H3 of the protruding portion 27 in the straight tube portion 23 and the height H2 of the protruding portion 27 in the proximal-side tapered portion 22 may be the same, that is, H3=H2, but the height H3 of the protruding portion 27 in the straight tube portion 23 may be higher than the height H2 of the protruding portion 27 in the proximal-side tapered portion 22, that is, H3>H2. As a result, the outer diameter of the balloon 20 at the proximal portion can be kept small, and thus, the maneuverability of the balloon 20 can be improved.

Even if the height H2 of the protruding portion 27 in the proximal-side tapered portion 22 is relatively low, since the value of the ratio D2/H2 of the depth D2 of the PT-portion notch 28PT of the protruding portion 27 in the proximal-side tapered portion 22 to the height H2 of the protruding portion 27 in the proximal-side tapered portion 22 is relatively small, the rigidity of the protruding portion 27 in the proximal-side tapered portion 22 can be improved, thereby improving the function of expanding a stenosed site when the balloon 20 is deflated.

As illustrated in FIG. 15, it is preferable that the height of the protruding portion 27 in the proximal-side tapered portion 22 gradually decrease from the distal side to the proximal side in the longitudinal axis direction x. In this case, it is also a preferable aspect that an inner protruding portion is formed in the proximal-side tapered portion 22. As a result, the rigidity of the portion of the balloon 20 on the proximal side can be further improved, and kinking and excessive inflation during pressurization can be suppressed.

Alternatively, as illustrated in FIG. 16, the relationship of D3/H3>D2/H2 may be satisfied in a state in which the height H3 of the protruding portion 27 in the straight tube portion 23 and the height H2 of the protruding portion 27 in the proximal-side tapered portion 22 satisfy the relationship of H3<H2. Accordingly, since the depth D2 of the PT-portion notch 28PT of the protruding portion 27, which has a relatively large height, in the proximal-side tapered portion 22 can be made relatively small, it is possible to improve the rigidity of the protruding portion 27 in the proximal-side tapered portion 22, thereby improving the function of expanding a stenosed site when the balloon 20 is deflated.

As illustrated in FIG. 16, the height of the protruding portion 27 in the proximal-side tapered portion 22 may gradually increase from the distal side to the proximal side in the longitudinal axis direction x. As a result, the rigidity of the portion of the balloon 20 on the proximal side can be improved, so that the balloon 20 can be supported, thereby stabilizing the maneuverability of the balloon 20. Further, in this case, the protruding portion 27 may be provided to extend from the proximal-side tapered portion 22 to the proximal-side sleeve portion 21, and the height of the protruding portion 27 in the proximal-side sleeve portion 21 may be higher than the height H2 of the protruding portion 27 in the proximal-side tapered portion 22. As a result, the protruding portion 27 in the proximal-side sleeve portion 21 can also act on a stenosed site when the balloon 20 is deflated, so that the function of expanding the stenosed site can be further improved. In addition, it is possible to further improve the rigidity of the portion of the balloon 20 on the proximal side. The proximal-side sleeve portion 21 may or may not be provided with at least one of the notches 28.

In the straight tube portion 23, the depth D3 of the ST-portion notch 28ST and the height H3 of the protruding portion 27 may satisfy a relationship of D3≥H3×¼. As a result, the ratio of the depth D3 of the ST-portion notch 28ST to the height H3 of the protruding portion 27 in the straight tube portion 23 can be set to a predetermined value or more, and thus, the function of expanding a stenosed site can be easily improved by the protruding portion segments 27S acting on the stenosed site.

In the distal-side tapered portion 24, the depth D4 of the DT-portion notch 28DT and the height H4 of the protruding portion 27 may satisfy a relationship of D4≥H4×¼. Accordingly, the ratio of the depth D4 of the DT-portion notch 28DT to the height H4 of the protruding portion 27 in the distal-side tapered portion 24 can be set to be equal to or greater than a predetermined value, and thus, the protruding portion segments 27S divided by the DT-portion notch 28DT can more easily act on a blood vessel wall when the balloon 20 is in the deflated state, so that, the function of expanding a stenosed site can be easily improved.

When the protruding portion 27 in the proximal-side tapered portion 22 has the PT-portion notch 28PT, the depth D2 of the PT-portion notch 28PT may be 50 μm or more, and the depth D2 of the PT-portion notch 28PT and the height H2 of the protruding portion 27 in the proximal-side tapered portion 22 may satisfy a relationship of D2≥H2×¼. As a result, the ratio of the depth D2 of the PT-portion notch 28PT to the height H2 of the protruding portion 27 in the proximal-side tapered portion 22 can be set to a predetermined value or more, and thus, the protruding portion segments 27S divided by the PT-portion notch 28PT can more easily act on a blood vessel wall, so that the function of expanding a stenosed site when the balloon 20 is deflated can be easily improved.

The number of the ST-portion notches 28ST in the straight tube portion 23 may be larger than the number of the DT-portion notches 28DT in the distal-side tapered portion 24. In this case, even when the depth D3 of each ST-portion notch 28ST in the straight tube portion 23 is relatively small, the function of expanding a stenosed site by each ST-portion notch 28ST can be improved when the balloon 20 is inflated. In addition, since the number of the DT-portion notches 28DT in the distal-side tapered portion 24 is relatively small, the rigidity of the protruding portion 27 in the distal-side tapered portion 24 can be improved, and the function of expanding a stenosed site when the balloon 20 is deflated can be improved.

When the protruding portion 27 in the proximal-side tapered portion 22 has the PT-portion notch 28PT, the depth D2 of the PT-portion notch 28PT may be 50 μm or more, and the number of the ST-portion notches 28ST in the straight tube portion 23 may be larger than the number of the PT-portion notches 28PT in the proximal-side tapered portion 22. Since the straight tube portion 23 is a long portion of the balloon 20 in the longitudinal axis direction x, having a relatively large number of the ST-portion notches 28ST in the straight tube portion 23 makes it easy to ensure the function of expanding a stenosed site by the ST-portion notches 28ST when the balloon 20 is inflated. In addition, since the number of the PT-portion notches 28PT in the proximal-side tapered portion 22 is relatively small, the rigidity of the protruding portion 27 in the proximal-side tapered portion 22 can be improved, thereby improving the function of expanding a stenosed site when the balloon 20 is deflated.

The number of the ST-portion notches 28ST in the straight tube portion 23 may be 1 or more, 3 or more, or 5 or more, and 20 or less, 16 or less, 12 or less, or 8 or less. The number of the DT-portion notches 28DT in the distal-side tapered portion 24 may be 1, and may be 2 or more, 3 or more, and 10 or less, 7 or less, or 5 or less. The number of the PT-portion notches 28PT in the proximal-side tapered portion 22 may be 1, and may be 2 or more, 3 or more, and 10 or less, 7 or less, or 5 or less.

The balloon main body 26 may be made of a resin. The resin may be a thermoplastic resin. This makes it easy to manufacture the balloon 20 by molding. Examples of the resin forming the balloon main body 26 include polyolefin-based resins such as polyethylene, polypropylene, and ethylene-propylene copolymers, polyester-based resins such as polyethylene terephthalate and polyester elastomers, polyurethane-based resins such as polyurethane and polyurethane elastomers, polyphenylene sulfide resins, polyamide-based resins such as polyamides and polyamide elastomers, fluororesins, silicone-based resins, and natural rubbers such as latex. Only one of these may be used, or two or more of these may be used in combination. Among the above, polyamide-based resins, polyester-based resins, and polyurethane-based resins are suitably used. In particular, it is preferable to use an elastomer resin from the viewpoint of thinning and flexibility of the balloon 20. Examples of a material that is suitable for the balloon 20 among the polyamide-based resins include nylon 12 and nylon 11, and nylon 12 is suitably used from the viewpoint of being relatively easily moldable during blow molding. In addition, from the viewpoint of thinning and flexibility of the balloon 20, polyamide elastomers such as polyether-ester-amide elastomer and polyamide ether elastomer may be used. In particular, polyether-ester-amide elastomer may be used from the viewpoint of high yield strength and favorable dimensional stability of the balloon 20.

The protruding portion 27 can be made of, for example, a resin. When the protruding portion 27 is made of a resin, the protruding portion 27 and the balloon main body 26 may be made of the same resin, and the protruding portion 27 and the balloon main body 26 may be integrally formed. That is, instead of the protruding portion 27 being attached to the outer side surface of the balloon main body 26, it is preferable that a thin portion of the balloon 20 form the balloon main body 26 and that a thick portion of the balloon 20 form the protruding portion 27. The balloon main body 26 may have an inner layer and an outer layer. In this case, the protruding portion 27 may be made of the same resin as that of the outer layer of the balloon main body 26. With such a configuration, the protruding portion 27 is less likely to unintentionally fall off from the balloon main body 26. Alternatively, the protruding portion 27 and the balloon main body 26 may be formed of different resins as long as the resin forming the protruding portion 27 and the resin forming the balloon main body 26 have a certain degree of compatibility. The protruding portion 27 may be attached as a separate member to the outer side surface of the balloon main body 26 by means of welding, adhesive bonding, or the like.

The protruding portion 27 may be made of a metal or a combination of a metal and a resin. The protruding portion 27 may be attached as a separate member to the outer side surface of the balloon main body 26 by means of welding, adhesive bonding, or the like.

### 2. Balloon Catheter

The balloon catheter 10 according to one or more embodiments of the present invention includes the above-described balloon 20 for a balloon catheter. As described in the above section “1. Balloon for Balloon Catheter”, the balloon 20 is connected to the distal end portion of the shaft 30 as illustrated in FIG. 1.

FIG. 1 illustrates the so-called rapid-exchange balloon catheter 10 that includes a guidewire port 31a formed at an intermediate portion of the shaft 30 in a direction from the distal side toward the proximal side, and an inner shaft 31 serving as a guidewire passage extending from the guidewire port 31a to the distal side of the shaft 30.

The shaft 30 may have a fluid flow path and the guidewire passage therein. In order for the shaft 30 to have the fluid flow path and the guidewire passage therein, for example, the shaft 30 may be configured such that it includes the inner shaft 31 and the outer shaft 32 disposed outside the inner shaft 31, the inner shaft 31 functioning as the guidewire passage, and such that a space between the outer shaft 32 and the inner shaft 31 functions as the fluid flow path. In the case of such a configuration, it is preferable that the inner shaft 31 extend to the distal side by penetrating the balloon 20 such that the distal side of the balloon 20 is connected to the inner shaft 31 and such that the proximal side of the balloon 20 is connected to the outer shaft 32.

The balloon catheter 10 may have a distal-side outer shaft 32d and a proximal-side outer shaft 32p. The distal-side outer shaft 32d and the proximal-side outer shaft 32p may be separate members, and a proximal end portion of the distal-side outer shaft 32d may be connected to a distal end portion of the proximal-side outer shaft 32p so as to constitute the outer shaft 32 extending from the balloon 20 to a proximal end portion of the balloon catheter 10. Alternatively, the single outer shaft 32 may extend from the balloon 20 to the proximal end portion of the balloon catheter 10, and the distal-side outer shaft 32d or the proximal-side outer shaft 32p may further be formed of a plurality of tube members.

The shaft 30 may be made of a resin, a metal, or a combination of a resin and a metal. By using a resin as the material forming the shaft 30, it becomes easy to impart flexibility and elasticity to the shaft 30. In addition, by using a metal as the material forming the shaft 30, the deliverability of the balloon catheter 10 can be improved.

Examples of a resin forming the shaft 30 include polyamide-based resins, polyester-based resins, polyurethane-based resins, polyolefin-based resins, fluororesins, vinyl chloride-based resins, silicone-based resins, natural rubbers, and synthetic rubbers. Only one of these may be used, or two or more of these may be used in combination. Examples of a metal forming the shaft 30 include stainless steels such as SUS 304 and SUS 316, platinum, nickel, cobalt, chromium, titanium, tungsten, gold, Ni—Ti alloys, Co—Cr alloys, or combinations thereof. In the case where the shaft 30 includes the distal-side outer shaft 32d and the proximal-side outer shaft 32p, which are separate members, for example, the distal-side outer shaft 32d can be made of a resin, and the proximal-side outer shaft 32p can be made of a metal. Further, the shaft 30 may have a multilayer structure made of different materials or the same material.

The balloon 20 and the shaft 30 may be joined to each other by, for example, bonding with an adhesive, welding, or crimping with a ring-shaped member attached to a portion where an end portion of the balloon 20 and the shaft 30 overlap each other. In particular, the balloon 20 and the shaft 30 may be joined to each other by welding. By welding the balloon 20 and the shaft 30 to each other, even when the balloon 20 is repeatedly inflated or deflated, the joint between the balloon 20 and the shaft 30 is less likely to be released, and the joint strength can be improved.

A distal end portion of the balloon catheter 10 may be provided with a tip member 60. The tip member 60 may be provided at the distal end portion of the balloon catheter 10 by being connected to a distal end portion of the balloon 20 as a separate member from the inner shaft 31, or a portion of the inner shaft 31 that extends further toward the distal side than the distal end portion of the balloon 20 may function as the tip member 60.

A radiopaque marker 70 may be disposed at a position on the shaft 30 where the balloon 20 is located in the longitudinal axis direction x, in order to enable confirmation of the position of the balloon 20 under X-ray fluoroscopy. For example, the radiopaque marker 70 can be disposed on the inner shaft 31 disposed inside the balloon 20. The radiopaque marker 70 may be disposed at positions corresponding to both ends of the straight tube portion 23 of the balloon 20, and may alternatively be disposed at a position corresponding to the center of the straight tube portion 23 of the balloon 20.

A hub 40 may be provided on the proximal side of the shaft 30, and the hub 40 may be provided with a fluid injection portion 50 that communicates with the flow path of the fluid to be supplied to the inside of the balloon 20.

The shaft 30 and the hub 40 are joined to each other by, for example, bonding with an adhesive or welding. In particular, the shaft 30 and the hub 40 may be bonded to each other by adhesive bonding. Since the shaft 30 and the hub 40 are bonded to each other, for example, in the case where a material forming the shaft 30 and a material forming the hub 40 are different from each other, such as the case where the shaft 30 is formed of a material having high flexibility while the hub 40 is formed of a material having high rigidity, the bonding strength between the shaft 30 and the hub 40 can be increased, thereby improving the durability of the balloon catheter 10.

Although not illustrated, one or more embodiments of the present invention can also be applied to a so-called over-the-wire balloon catheter having a guidewire passage extending from the distal side to the proximal side of a shaft. In the case of an over-the-wire balloon catheter, it is preferable that an inflation lumen and a guidewire lumen extend to a hub disposed on the proximal side and that proximal-side openings of the respective lumens be provided in the hub having a bifurcated structure.

In the case of a rapid-exchange catheter, it is preferable that the outer wall of the distal-side outer shaft 32d and/or the outer wall of the proximal-side outer shaft 32p be appropriately coated, and it is more preferable that both the distal-side outer shaft 32d and the proximal-side outer shaft 32p be coated. In the case of an over-the-wire catheter, it is preferable that an outer wall of an outer shaft be appropriately coated.

The coating can be a hydrophilic coating or a hydrophobic coating depending on the purpose, and can be applied by immersing the shaft 30 in a hydrophilic coating agent or a hydrophobic coating agent, applying a hydrophilic coating agent or a hydrophobic coating agent to the outer wall of the shaft 30, or covering the outer wall of the shaft 30 with a hydrophilic coating agent or a hydrophobic coating agent. The coating agent may contain a drug or an additive.

Examples of the hydrophilic coating agent include hydrophilic coating agents made of hydrophilic polymers such as polyvinyl alcohol, polyethylene glycol, polyacrylamide, polyvinylpyrrolidone, and methyl vinyl ether-maleic anhydride copolymer, or any combination thereof.

Examples of the hydrophobic coating agent include polytetrafluoroethylene (PTFE), fluorinated ethylene propylene (FEP), perfluoroalkoxyalkane (PFA), silicone oil, hydrophobic urethane resin, carbon coating, diamond coating, diamond-like carbon (DLC) coating, ceramic coating, and substances having low surface free energy terminated with alkyl groups or perfluoroalkyl groups.

A drug may be retained on the outer surface of the straight tube portion 23 of the balloon 20. The drug is not particularly limited as long as it is a pharmacologically active substance, and examples of such drugs include pharmaceutically acceptable drugs, such as gene therapy agents, non-gene therapy agents, small molecules, and cells. In particular, when a catheter is used for the purpose of suppressing restenosis of a blood vessel after treatment in angioplasty, an anti-restenosis agent, such as an antiproliferative agent or an immunosuppressive agent, may be used as the drug. Examples of such drugs include paclitaxel, sirolimus (rapamycin), everolimus, and zotarolimus.

This application claims the benefit of priority based on Japanese Patent Application No. 2023-89136, filed on May 30, 2023. The entire disclosure of Japanese Patent Application No. 2023-89136, filed on May 30, 2023, is incorporated herein by reference in its entirety.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

### REFERENCE SIGNS LIST

10 balloon catheter
20 balloon for balloon catheter
21 proximal-side sleeve portion
22 proximal-side tapered portion
23 straight tube portion
24 distal-side tapered portion
25 distal-side sleeve portion
26 balloon main body
27 protruding portion
27b base portion of protruding portion
27t top portion of protruding portion
27S protruding portion segment
28 notch
28b bottom portion of notch
28t top portion of notch
28DT DT-portion notch
28PT PT-portion notch
28ST ST-portion notch
29 wing
29a wing forming portion
30 shaft
31 inner shaft
31a guidewire port
32 outer shaft
32d distal-side outer shaft
32p proximal-side outer shaft
40 hub
50 fluid injection portion
60 tip member
70 radiopaque marker
D2 depth of notch in proximal-side tapered portion
D3 depth of notch in straight tube portion
D4 depth of notch in distal-side tapered portion
H2 height of protruding portion in proximal-side tapered portion
H3 height of protruding portion in straight tube portion
H4 height of protruding portion in distal-side tapered portion

1. A balloon for a balloon catheter comprising:
a straight tube portion;
a proximal-side tapered portion positioned on a proximal side of the straight tube portion;
a proximal-side sleeve portion positioned on a proximal side of the proximal-side tapered portion;
a distal-side tapered portion positioned on a distal side of the straight tube portion;
a distal-side sleeve portion positioned on a distal side of the distal-side tapered portion;
a balloon main body having an outer surface and an inner surface; and
a protruding portion protruding radially outward from the outer surface of the balloon main body and extending in a longitudinal axis direction,
wherein the protruding portion in the straight tube portion has at least one notch (ST-portion notch), and the protruding portion in the distal-side tapered portion has at least one notch (DT-portion notch),
wherein a depth D3 of the at least one ST-portion notch and a depth D4 of the at least one DT-portion notch are each 50 μm or more, and
wherein the depth D3 of the at least one ST-portion notch, a height H3 of the protruding portion in the straight tube portion, the depth D4 of the at least one DT-portion notch, and a height H4 of the protruding portion in the distal-side tapered portion satisfy a relationship of D3/H3≤D4/H4.

2. The balloon for the balloon catheter according to claim 1, wherein the at least one ST-portion notch includes a plurality of ST-portion notches, and the depth D3 is an average of depths of the plurality of ST-portion notches.

3. The balloon for the balloon catheter according to claim 1, wherein the at least one DT-portion notch includes a plurality of DT-portion notches, and the depth D4 is an average of depths of the plurality of DT-portion notches.

4. The balloon for the balloon catheter according to claim 1, wherein the height H3 of the protruding portion in the straight tube portion and the height H4 of the protruding portion in the distal-side tapered portion satisfy a relationship of H3≥H4.

5. The balloon for the balloon catheter according to claim 1, wherein:
the protruding portion in the proximal-side tapered portion has at least one notch (PT-portion notch),
a depth D2 of the at least one PT-portion notch is 50 μm or more, and
the depth D3 of the at least one ST-portion notch, the height H3 of the protruding portion in the straight tube portion, the depth D2 of the at least one PT-portion notch, and a height H2 of the protruding portion in the proximal-side tapered portion satisfy a relationship of D3/H3≤D2/H2.

6. The balloon for the balloon catheter according to claim 5, wherein the at least one PT-portion notch includes a plurality of PT-portion notches, and the depth D2 is an average of depths of the plurality of PT-portion notches.

7. The balloon for the balloon catheter according to claim 5, wherein the height H3 of the protruding portion in the straight tube portion and the height H2 of the protruding portion in the proximal-side tapered portion satisfy a relationship of H3≥H2.

8. The balloon for the balloon catheter according to claim 5, wherein the height H3 of the protruding portion in the straight tube portion and the height H2 of the protruding portion in the proximal-side tapered portion satisfy a relationship of H3<H2.

9. The balloon for the balloon catheter according to claim 1, wherein:
the protruding portion in the proximal-side tapered portion has a notch (PT-portion notch),
a depth D2 of the PT-portion notch is 50 μm or more, and
the depth D3 of the at least one ST-portion notch, the height H3 of the protruding portion in the straight tube portion, the depth D2 of the PT-portion notch, and a height H2 of the protruding portion in the proximal-side tapered portion satisfy a relationship of D3/H3>D2/H2.

10. The balloon for the balloon catheter according to claim 9, wherein the height H3 of the protruding portion in the straight tube portion and the height H2 of the protruding portion in the proximal-side tapered portion satisfy a relationship of H3≥H2.

11. The balloon for the balloon catheter according to claim 9, wherein the height H3 of the protruding portion in the straight tube portion and the height H2 of the protruding portion in the proximal-side tapered portion satisfy a relationship of H3<H2.

12. The balloon for the balloon catheter according to claim 1, wherein the depth D3 of the at least one ST-portion notch and the height H3 of the protruding portion in the straight tube portion satisfy a relationship of D3≥H3×¼.

13. The balloon for the balloon catheter according to claim 1, wherein the depth D4 of the at least one DT-portion notch and a height H4 of the protruding portion in the distal-side tapered portion satisfy a relationship of D4≥H4×¼.

14. The balloon for the balloon catheter according to claim 1, wherein:
the protruding portion in the proximal-side tapered portion has a notch (PT-portion notch),
a depth D2 of the PT-portion notch is 50 μm or more, and
the depth D2 of the PT-portion notch and a height H2 of the protruding portion in the proximal-side tapered portion satisfy a relationship of D2≥H2×¼.

15. The balloon for the balloon catheter according to claim 1, wherein the at least one ST-portion notch is greater in number than the at least one DT-portion notch.

16. The balloon for the balloon catheter according to claim 1, wherein:
the protruding portion in the proximal-side tapered portion has a notch (PT-portion notch),
a depth D2 of the PT-portion notch is 50 μm or more, and
the at least one ST-portion notch is greater in number than the PT-portion notch.

17. A balloon catheter comprising the balloon for a balloon catheter according to claim 1.
